(19) United States
(12) Patent Application Publication
(10) Pub. No.: US 2014/0073916 A1
(43) Pub. Date: Mar. 13, 2014

(54) DEVICE FOR ASSISTING IN DETECTING ANATOMICAL FEATURES OF AT LEAST A PORTION OF A TISSUE AND METHOD FOR ASSISTING IN DETECTING ANATOMICAL FEATURES OF AT LEAST A PORTION OF A TISSUE

(71) Applicants: Michel Paques, Paris (FR); Sarah Mrejen, Paris (FR)

(72) Inventors: Michel Paques, Paris (FR); Sarah Mrejen, Paris (FR)

(73) Assignee: UNIVERSITE PIERRE ET MARIE CURIE (PARIS 6), Paris (FR)

(21) Appl. No.: 14/111,466

(22) PCT Filed: Apr. 10, 2012
(86) PCT No.: PCT/EP2012/056428

§ 371 Date: Nov. 19, 2013

(30) Foreign Application Priority Data
Apr. 15, 2011 (FR) 1153306

(51) Int. Cl. A61B 3/10

(52) U.S. Cl. CPC A61B 3/102
USPC 600/425

(57) ABSTRACT

The invention relates to a device for assisting in detecting anatomical features of at least a portion of a tissue, in particular a retinal tissue, the device comprising means for acquiring images of said portion using optical coherence tomography (OCT), which are taken at separate angles of incidence, and detection means for detecting, in a single area of the portion studied, at least one pattern having at least one feature that varies from one image to another, the pattern being attributable to the presence of anatomical features. The invention likewise relates to a method for assisting in detecting anatomical features of at least a portion of a tissue.

The invention relates to a device for aiding with detection of anatomical features in at least one portion of a tissue, for example a retinal tissue. The invention also relates to a method for aiding with detection of anatomical features in at least one portion of a tissue.

The retina is a thin stratified membrane that is a few tenths of a millimeter in thickness. FIG. 1 schematically shows a portion of a retina. Simplistically, the retina comprises three main layers. In the first layer, the retina comprises photoreceptors that have approximately the shape of rods 1 (for black-and-white and scotopic vision) or cones 2 (for color and photopic vision), the photoreceptors converting light signals into nerve impulses. These nerve impulses are then transmitted to what are called bipolar cells 3 located in the second layer. The bipolar cells 3 communicate the nerve impulses to what are called ganglion cells 4 located in the third layer. Axons of the ganglion cells 4 are connected to the brain in order to transmit the nerve pulses to it, all of said axons forming a nerve called the optical nerve. The three main layers are separated by two intermediate layers that comprise cells that participate in the regulation of the transmission of the nerve impulses. In the first intermediate layer, what are called horizontal cells 5 receive nerve impulses from the photoreceptors 1, 2 and transmit them to adjacent bipolar cells 3. In the second intermediate layer, what are called amacrine cells 6 receive nerve impulses from bipolar cells 3 and transmit them to adjacent ganglion cells 4.

Blood vessels are present within the retina in order to irrigate said retina. Furthermore, certain structures of the retina take the form of fibrils that lie substantially parallel to one another, such as the axons of the photoreceptors, the axons of the ganglion cells, and the external segments of the photoreceptors, etc.

### BACKGROUND OF THE INVENTION

Optical coherence tomography or OCT is a recent imaging technique that allows biological structures to be studied very precisely. OCT permits non-invasive examination of a portion of a tissue (retinal tissue, mucosal tissue, epidermal tissue, etc.).

Simplistically, OCT is carried out as follows: a narrow light beam, most often an infrared beam, emitted by a source is divided into two parts directed into two legs by a beam splitter. In one leg, the first part of the beam penetrates into a portion of the tissue to be studied in order to be backscattered by said portion. In the second leg, called the reference leg, the second part of the beam is reflected by a planar mirror. The two parts of the beam are then directed toward a Michelson interferometer that allows interference fringes to be extracted from the two parts of the beam. The interference fringes contain information on the studied tissue portion. Via a point-by-point sweep of the tissue studied by the method just described, the information gathered is combined by processing means that then produce a veritable optical cross section of said tissue, said cross section having a resolution of about a few microns.

In the field of ophthalmology, it is thus known to carry out an OCT examination in order to study a retina and especially the various layers of the retina. FIG. 2a (or FIG. 10) is an image taking during an OCT examination of a normal retina. FIG. 2b schematically illustrates a histological cross section of the same retina observed under a microscope. The various layers of the retina are broken down as follows:
the ganglion cell layer (GCL);
the inner plexiform layer (IPL);
the inner nuclear layer (INL);
the outer plexiform layer (OPL);
the Henle's fiber layer (HFL);
the outer nuclear layer (ONL);
the outer segment of the photoreceptor layer (OS/PR); and
the retinal pigment epithelium (RPE).

It has been observed that an OCT examination of the retina is much more precise than a conventional examination such as echography of the retina. An OCT examination may therefore prove to be critical for detecting a problem with a retina. For example, an OCT examination allows lesions to be seen that up to now could not be seen during conventional examinations. However, the lesions observed by OCT are very varied in nature thereby making it difficult to classify said lesions by their description. It therefore proves to be difficult to detect certain lesions automatically from images taken by OCT.

Generally, the interpretation of images taken by OCT relies on the intuition and experience of the doctor uniquely, and this results in a heavy workload for said doctor.

The article “Revealing Henle's Fiber Layer using Spectral Domain Optical Coherence Tomography” published on the Internet site of the journal Investigative Ophthalmology and Visual Science on 11 Nov. 2010 describes a method for precisely identifying the Henle's fiber layer (HFL), which layer especially contains the axons of the photoreceptors, during an OCT examination. Specifically, with a conventional OCT examination, it proves to be tricky to distinguish the outer nuclear layer (ONL) from the HFL. It is thus explained in said document that reflection from the HFL depends on the angle with which a beam penetrates the retina during an OCT examination, the ONL not exhibiting this distinctive trait. Via choice of a suitable angle of incidence, reflection from the HFL may be made to be very different from that of the ONL, thereby allowing them to be clearly differentiated. It is then even possible to measure the thickness of the HFL.

The article “Revealing Henle's Fiber Layer using Spectral Domain Optical Coherence Tomography” describes a method allowing an entire and particular layer of the retina, namely the HFL, to be clearly identified. This increases the amount of information that it is possible to obtain from images taken by OCT in the case of a study of a retinal tissue, thereby lightening the work to be done by the doctor interpreting the images.

### Subject of the Invention

One aim of the invention is to make it even simpler for a doctor to interpret images taken by OCT of a portion of a tissue, by aiding with the detection of anatomical features in said portion.

The invention is applicable to any type of tissue and especially retinal tissue.

### BRIEF DESCRIPTION OF THE INVENTION

With a view to achieving this aim, a device is provided for aiding with detection of anatomical features in at least one portion of a tissue, especially a retinal tissue, the device comprising means for acquiring, by optical coherence tomography (OCT), images of said portion taken at separate angles of incidence, and detecting means for detecting in the images, in a given region of the studied portion, at least one pattern having at least one characteristic that varies from one image to another, the pattern being liable to be due to the presence of anatomical features.

The inventors have observed that two images taken at separate angles of incidence may exhibit patterns, each having at least one characteristic that varies from one image to another. Comparison and analysis of the two images thus aids with detection of anatomical features possibly related to such patterns.

The device according to the invention increases the amount of information that it is possible to obtain from images taken by OCT, thereby simplifying the work done by the doctor interpreting the images. The doctor may concentrate on the meaning of the patterns, their link with anatomical features or even whether said anatomical features are linked with an eventual pathology of the portion of tissue studied.

From one image to another, it is indeed the same pattern that is registered. However, said pattern is only slightly different from one image to another because of the variation in at least one of its characteristics. For example, the characteristic may be the color of the pattern, the shape of the pattern, the orientation of the pattern, the reflectance of the pattern, etc.

A method is also provided for aiding with detection of anatomical features in at least one portion of a tissue, the method comprising steps of:
acquiring, by optical coherence tomography, at least two images of said portion taken at separate angles of incidence; and
detecting in the images, in a given region of the studied portion, at least one pattern having at least one characteristic that varies from one image to another, the pattern being liable to be due to the presence of anatomical features.

### BRIEF DESCRIPTION OF THE DRAWINGS

FIGS. 1, 2a and 2b and 10 have already been described and show the structure of a retina. FIG. 10 is a negative (the balance of blacks and whites is inverted) of FIG. 2a.

The invention will be better understood in light of the following description of one particular nonlimiting embodiment of the invention, given with reference to the appended figures in which:

FIG. 3 is a schematic showing the various steps of the method of the invention;

FIGS. 4a and 4b each illustrate two images of a given portion of a retinal tissue, each view having been acquired by OCT at a separate angle of incidence, FIG. 4a illustrating the raw images output from an OCT examination, and FIG. 4b the images after reorientation;

FIG. 5a is a cross-sectional view of a retina;

FIG. 5b is an enlargement of a part of the cross section illustrated in FIG. 5a;

FIG. 5c is a schematic representation of the patterns present in the cross sections illustrated in FIGS. 5a and 5b;

FIG. 6 is a cross-sectional view of a retinal tissue of a diseased person;

FIG. 7 is a schematic showing the various steps of one particular implementation of the method of the invention;

FIG. 8 illustrates three images of a given portion of a retinal tissue, each view having been acquired by OCT at a separate angle of incidence; and

FIG. 9 illustrates three images of a given portion of a retinal tissue of a diseased person, each view having been acquired by OCT at a separate angle of incidence.

FIG. 11a is a negative view of FIG. 4a.

FIG. 11b is a negative view of FIG. 4b.

FIG. 12a is a negative view of FIG. 5a.

FIG. 12b is a negative view of FIG. 5b.

FIG. 13 is a negative view of FIG. 6.

FIG. 14 is a negative view of FIG. 7.

FIG. 15 is a negative view of FIG. 8.

FIG. 16 is a negative view of FIG. 9.

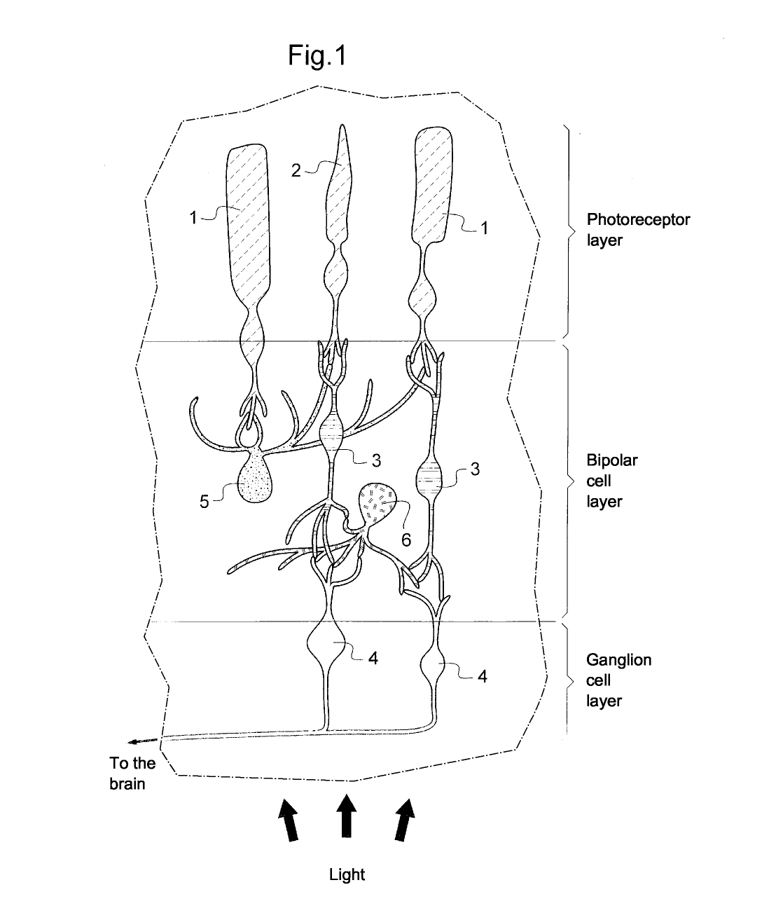

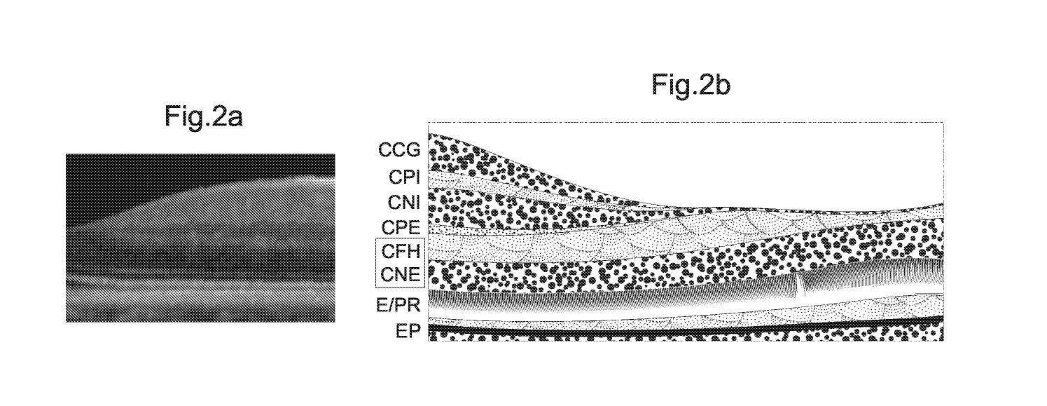

### DETAILED DESCRIPTION OF THE INVENTION

The device and method for aiding with detection of anatomical features of the invention are here applied to a portion of a retinal tissue. Of course, this application is nonlimiting.

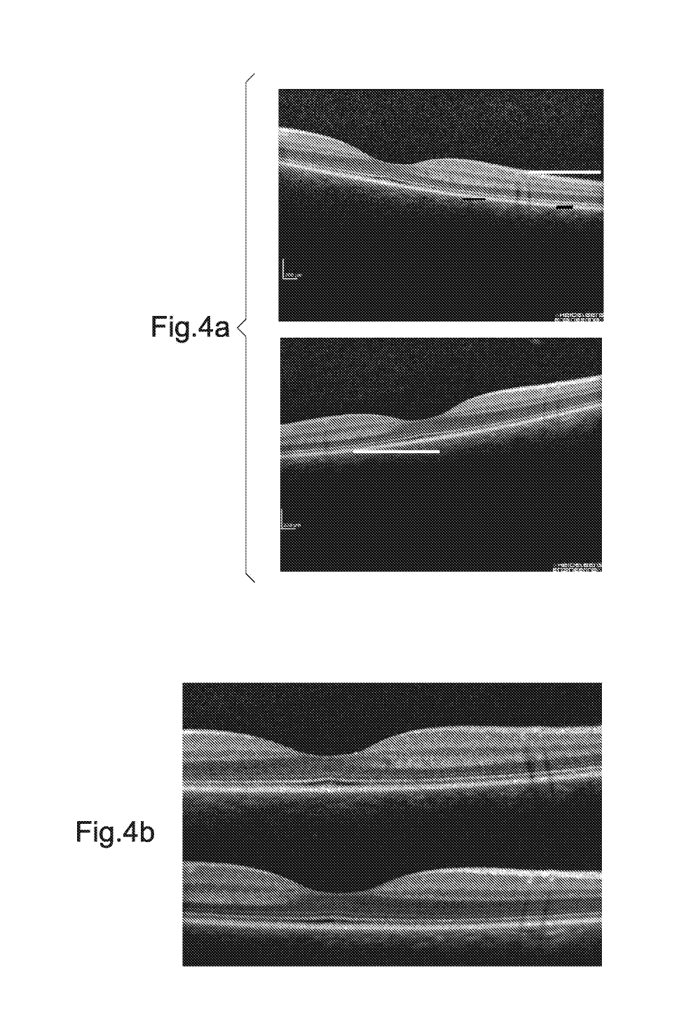

According to the invention, at least two images are acquired by optical coherence tomography (OCT) at separate angles of incidence. For the present invention, the images illustrated in FIG. 4a are raw images output from an OCT examination, and it is recommended to reorient them in one and the same direction (as illustrated in FIG. 4b) before they are compared.

The device according to the invention comprises means for acquiring images of said portion by optical coherence tomography (OCT), and detecting means.

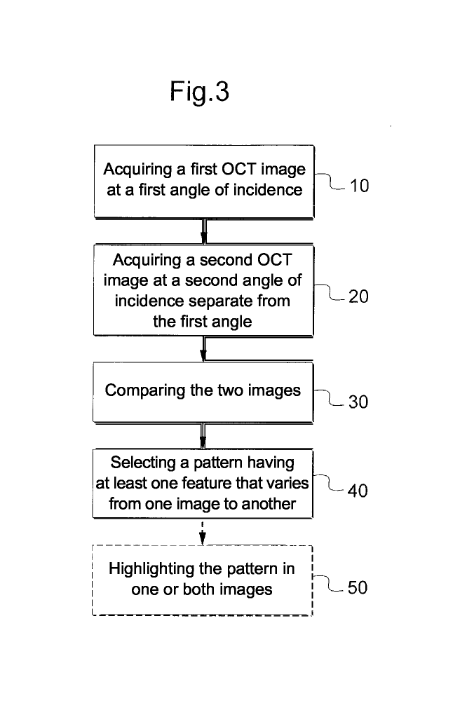

With reference to FIG. 3, in a first step 10 of the method according to the invention, the acquiring means of the device of the invention acquire a first optical coherence tomography or OCT image of a portion of a retinal tissue. Said first image is taken at a first angle of incidence. In a second operation, in a second step 20, the acquiring means of the device of the invention acquire a second OCT image of the same portion of retinal tissue, the second image being taken at a second angle of incidence separate from the first angle of incidence. As is known per se, in the case of manual acquisition of the images by a doctor, it turns out to be very easy to acquire images at separate angles of incidence. Specifically, the doctor must simply incline a control stick to select the angle of incidence with which they desire the image to be acquired.

The two images are then compared in a third step 30. In a fourth step 40, the detecting means detect in the two images, in a given region of the studied portion, the presence of a pattern having at least one characteristic that varies from one image to another.

In one particular embodiment, the device comprises means for highlighting the pattern. In a first embodiment, the highlighting means comprise means for encircling the detected pattern. In a second embodiment, the highlighting means comprise means for coloring the pattern. Thus, in a fifth step 50 of the method of the invention, the pattern is highlighted in each image or just in one of the images. The fifth step 50 thus makes it easy, even for a person not trained in medicine, to see said pattern.

The inventors have been able to demonstrate that such a pattern is liable to be due to the presence of anatomical features. The various steps 10, 20, 30, 40, 50 of the method described above thus make it possible to aid with the detection of such anatomical features.

Preferably, if no pattern is detected, another portion of the retinal tissue is analyzed by repeating the various steps described above starting with the acquiring first step 10.

In a first embodiment, in the fourth step 40, a pattern oriented differently from one image to another is more particularly sought. The characteristic of the pattern that varies is therefore here the orientation of the pattern. For this purpose, the detecting means of the device of the invention comprise means for searching for a pattern oriented differently from one image to another.

Specifically, the inventors were able to identify patterns, liable to be due to the presence of anatomical features, the orientation of which is entirely dependent on the angle of incidence at which the OCT images are acquired. This makes detection of said patterns much easier.

Even though the Henle's fiber layer or HFL appears different from one image to another because of its optical properties, it remains oriented in the same way and therefore does not hinder detection of the patterns.

In the case of a retinal tissue, on average, each angle of incidence may then be chosen in a range from about −20 to +20 degrees relative to the direction of a light beam passing through the retina during the OCT examination. The size of the eye studied and the diameter of the associated pupil may modify this range.

In a preferred embodiment of this first embodiment, in the fourth step 40, a pattern that is oriented differently from one image to another, and that is always oriented identically relative to the direction of a light beam that passes through the retina, is more particularly sought. For this purpose, the detecting means of the device of the invention comprise means for searching for a pattern oriented differently from one image to another, and oriented identically relative to the direction of a light beam that passes through the retina.

The inventors have been able to demonstrate that the interaction of blood vessels or red blood cells with the light beam that passes through the retina during the OCT examination could explain the presence of such patterns. Detection of said patterns will thus allow blood vessels or red blood cells to be located.

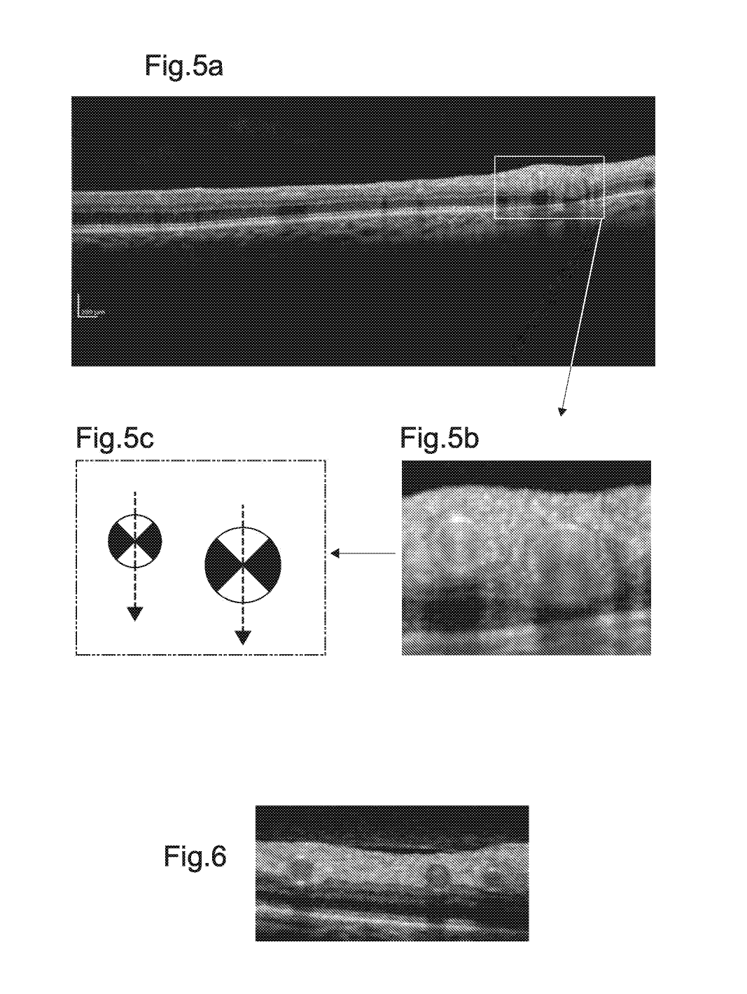

As a first variant of this preferred embodiment, an hourglass-shaped pattern is more particularly sought. For this purpose, the searching means comprise means for identifying an hourglass-shaped pattern. As illustrated in FIGS. 5a, 5b and 5c, two separate hourglasses are identifiable in one of the layers of the retinal tissue. As schematically shown in FIG. 5c, the orientation of the hourglasses is set by the angle of incidence with which the light beam (symbolized by the dotted arrows) passes through the retina during the OCT examination. An hourglass pattern therefore has a very particular appearance: two highly reflective regions (symbolized in white in FIG. 5c) are symmetrically located on either side of the light beam. These two regions are separated by two other darker regions also symmetrically located on either side of the light beam.

Thus, when the angle of incidence with which the second image is acquired is modified, the orientation of the hourglasses consequently changes so that the hourglasses are always oriented in the same way relative to the light beam.

From one image to another, only the hourglasses are oriented differently, thereby making the step of detecting said hourglasses easier.

The inventors were able to demonstrate that the hourglass patterns are liable to be due to the presence of blood vessels, such an hourglass being suggestive of the cross section of a blood vessel. When a light beam passes through a vessel, it is refracted by the walls of said vessel, thereby giving rise to a particular hour-glass-shaped representation.

As a second variant of this preferred embodiment, a pattern that is a shadow i.e. substantially a straight line the reflectance of which is much lower than a part of the surrounding tissue, is more particularly sought. For this purpose, the searching means comprise means for identifying a pattern that is a shadow. The orientation of the shadow depends on the angle of incidence. If the angle of incidence changes, there is a corresponding variation in the orientation of the shadow so that it is always parallel to the light beam. From one image to another, only the shadow is oriented differently, thereby making the step of detecting said shadow easier.

The inventors were able to demonstrate that patterns that are shadows are mainly the shadows of blood vessels.

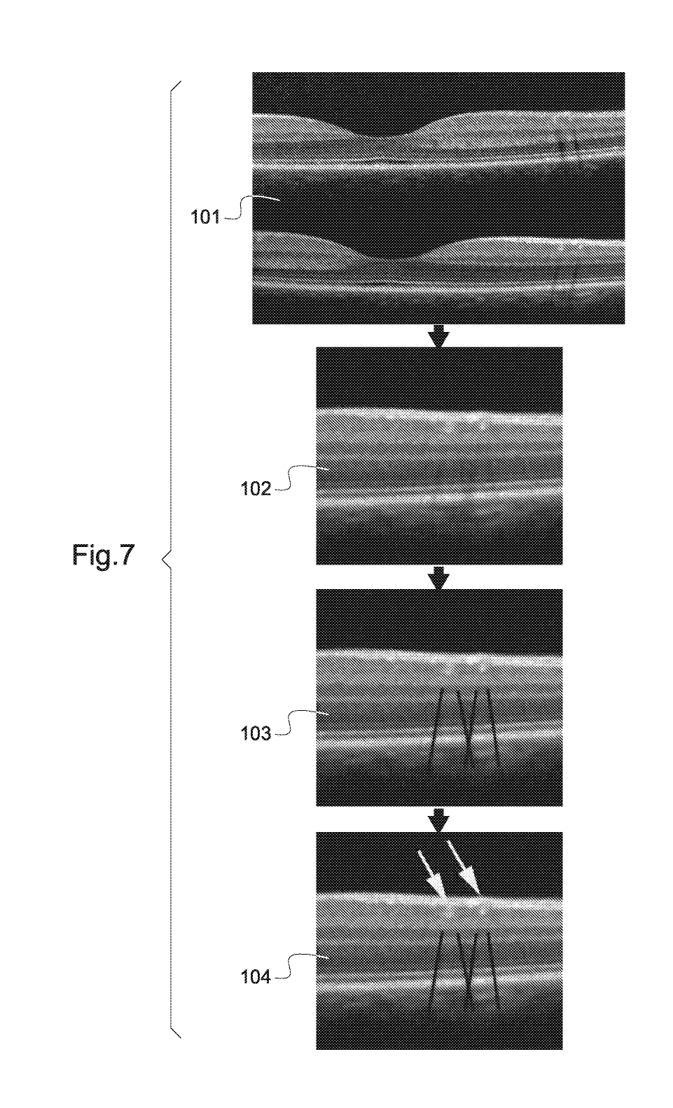

In one particular embodiment of this second variant, the device according to the invention furthermore comprises means for locating a focal point. With reference to FIG. 7, in a first step 101, two images taken by OCT at separate angles of incidence, and in which images two shadows have been detected, are selected. As may be seen, each shadow is oriented differently from one image to another, a shadow always being oriented in the same way relative to the light beam.

In a second step 102, superposing means of the device of the invention superpose the two images. Next, in a third step 103, the locating means extend the shadows. This step 103 is also called a triangulation step. Since the shadows are oriented differently from one image to another, their extensions end up converging. In the fourth step 104, the locating means identify the convergence point, which is the focal point.

The inventors have been able to demonstrate that generally said focal point is substantially near or even coincident with a blood vessel at the base of the shadow. Thus, by identifying the focal point, it is possible to identify an hourglass-shaped pattern near or coincident with said focal point, which pattern is representative of said blood vessel.

Thus, not only do the device and method of the invention allow a pattern that is a shadow to be detected in the images, but they also allow the blood vessel that is the root cause of the shadow to be localized.

The inventors have been able to demonstrate that it is easier to directly detect hourglass-shaped patterns that are liable to be due to the presence of large blood vessels. Regarding hourglass-shaped patterns that are liable to be due to the presence of small blood vessels, the inventors have been able to demonstrate that it is easier to detect said hourglass-shaped patterns by detecting the associated shadow patterns.

The device and method of the invention thus greatly simplify the task of a doctor who has to interpret said images.

The inventors have also demonstrated that the locating means might locate no blood vessel at the base of the shadow, and that the latter must consequently be due to the presence of another anatomical feature. More generally, it is therefore advantageous to search for a shadow in order to be able to localize an anatomical feature at the base of the shadow, said anatomical feature possibly being small in size. However, this situation is rarer and is only encountered in the case of a diseased retina, for example in the case of a retinal hemorrhage, an exudation, etc.

Moreover, the inventors have discovered that the refraction of a light beam by the vessel is directly related to the presence and the concentration of red blood cells in said vessel. Specifically, the red blood cells exert a high refractive power on the light beam. The hourglass-shaped pattern is therefore representative of the concentration of red blood cells in the associated blood vessel. FIG. 6 illustrates a retina of a person with anemia. The hourglass-shaped pattern is here clearly less visible than for a normal retina (FIGS. 5a, 5b and 5c). Thus, if the searching means cannot identify an hourglass-shaped pattern but only a shadow pattern, it is possible to follow the shadow to a focal point. A doctor studying said focal point may deduce that the hourglass-shaped pattern is not present or is very subdued, indicating a low red blood cell content and/or impaired blood circulation.

In one particular embodiment of this second variant, the device according to the invention furthermore comprises means for removing the shadow in order to create a new image in which the shadow no longer appears or only a marginal shadow appears, independently of whether this shadow is due to a blood vessel or to another anatomical feature.

Specifically, from one image to another, the shadow sees its orientation change. Thus, from one image to another, a section of a region of the retina containing the shadow is exposed to and another section is in turn masked by the shadow. Thus, the shadow never entirely covers the same section of said region. The removing means therefore retrieve, from each acquired image, a section of said region that is not covered by the shadow, and superpose said sections in order to reconstruct the region without the shadow or with a marginal shadow.

The artificially reconstructed region is then reintegrated into an image of the studied portion. The new image thus modified is of a much higher quality than the images comprising the shadow as the region previously masked by the shadow is now visible.

In a preferred embodiment, independently of whether a blood vessel is identified by an hourglass or by a shadow, in the fifth step 50, the highlighting means comprise means for coloring the blood vessel in each image. Then, if another axial cross section of the retinal tissue is studied, the highlighting means once more color the blood vessel in the new images. The device of the invention furthermore comprises means for assembling the various images so that, by coloring the blood vessel in all of the cross sections imaged, an angiography of said retina is thus obtained.

In a second embodiment, in the fourth step 40, a pattern the reflectance of which is different from one image to another is more particularly sought. Therefore, the characteristic of the pattern that varies is here the reflectance of the pattern. For this purpose, the detecting means of the device of the invention comprise means for searching for a pattern the reflectance of which is different from one image to another.

The inventors have been able to identify patterns, liable to be due to the presence of anatomical features, the reflectance of which varies depending on the angle of incidence at which the OCT images are acquired. This makes detection of said patterns much easier.

The inventors have been able to demonstrate that said patterns exhibit an optical anisotropy, reflection from the patterns depending on angle of incidence. This explains why their reflectance varies with angle of incidence.

The inventors have demonstrated that said patterns may be found in any region of the retina but are most often located in the outer segment of the photoreceptor layer (OS/PR).

Even though the Henle's fiber layer or HFL appears different from one image to another because of its optical properties, it is an integral, continuous and distinct layer of the retina and therefore it can easily be distinguished from patterns located in localized regions of the studied portion.

In the case of a retinal tissue, on average, each angle of incidence may then be chosen in a range from about −20 to +20 degrees relative to the direction of a light beam passing through the retina during the OCT examination. The size of the eye studied and the diameter of the associated pupil may modify this range.

In a preferred embodiment, the coloring means take into consideration the reflectance of the pattern and color said pattern more or less strongly from one image to another in order thus to illustrate the influence of angle of incidence on the reflectance of said pattern.

Preferably, the device according to the invention furthermore comprises selecting means. Likewise, the method according to the invention comprises an additional step that consists in selecting the image in which the pattern is brightest by way of the selecting means.

Preferably, the device according to the invention comprises means for calculating an angle of incidence at which said pattern has a maximal reflectance. Specifically, the calculating means determine the reflectance of the pattern in each image, and associate it with the angle of incidence at which the image was acquired. Next, the calculating means estimate the angle of incidence at which said pattern has a maximal reflectance.

Of course, the greater the number of images acquired, the more accurate the angle of maximum reflectance estimated by the calculating means will be.

In one particular embodiment, a line-shaped pattern is more particularly sought. For this purpose, the searching means comprise means for identifying a pattern that is a line. The reflectance of said line depends on the angle of incidence. If the angle of incidence changes, there is a corresponding variation in the reflectance of the line. From one image to another, only the line has a different reflectance, thereby making the step of detecting the lines easier.

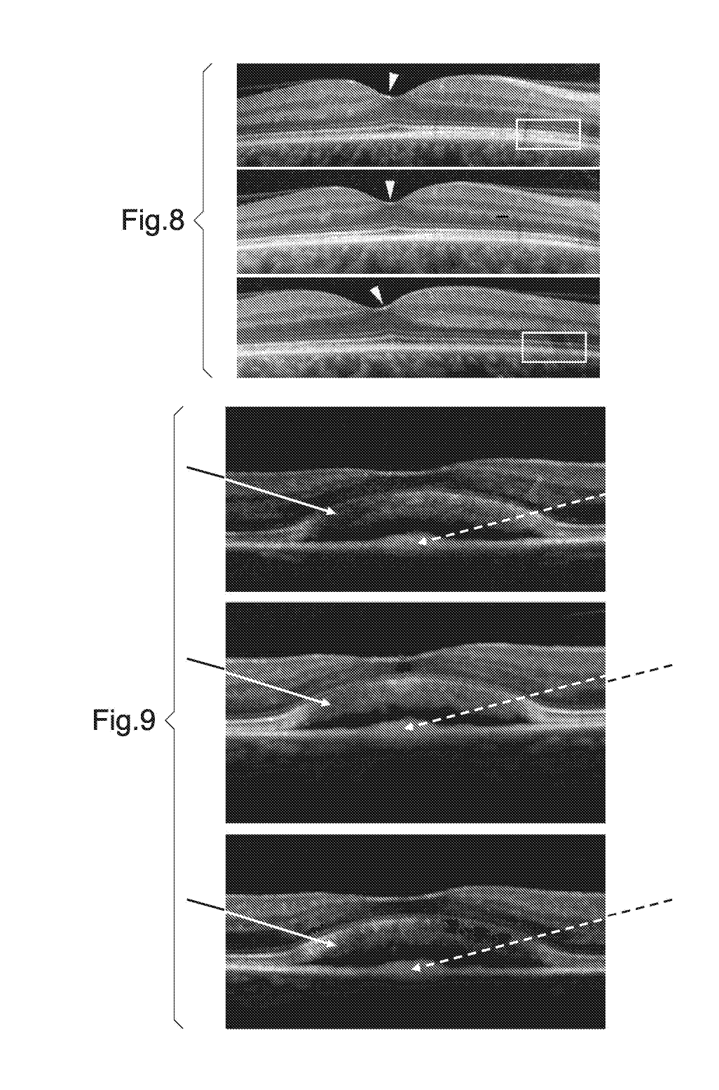

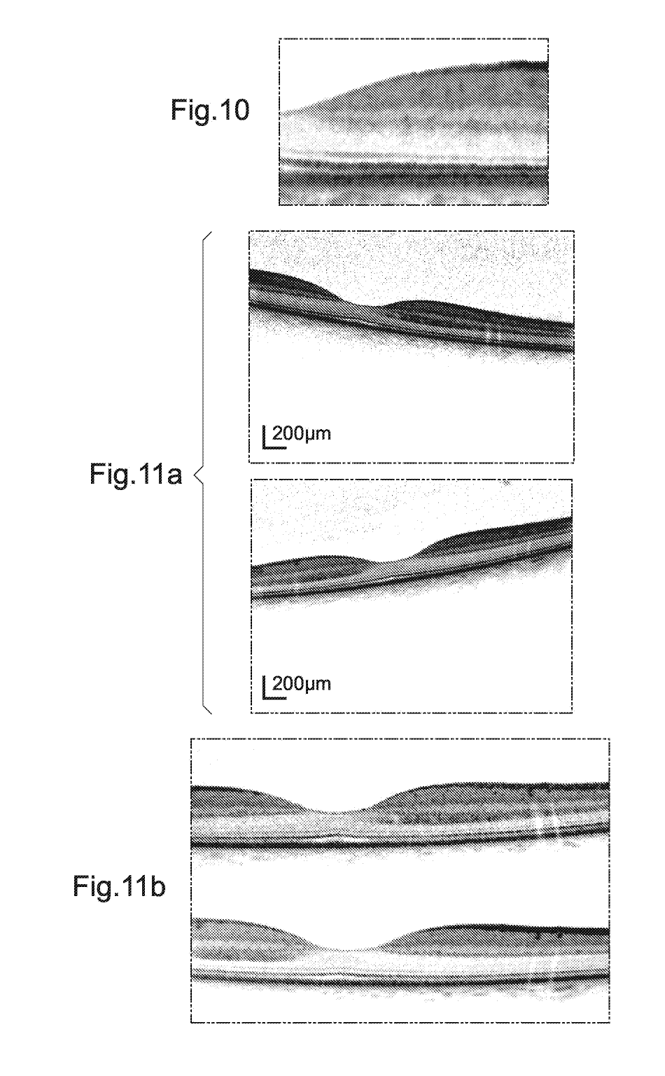

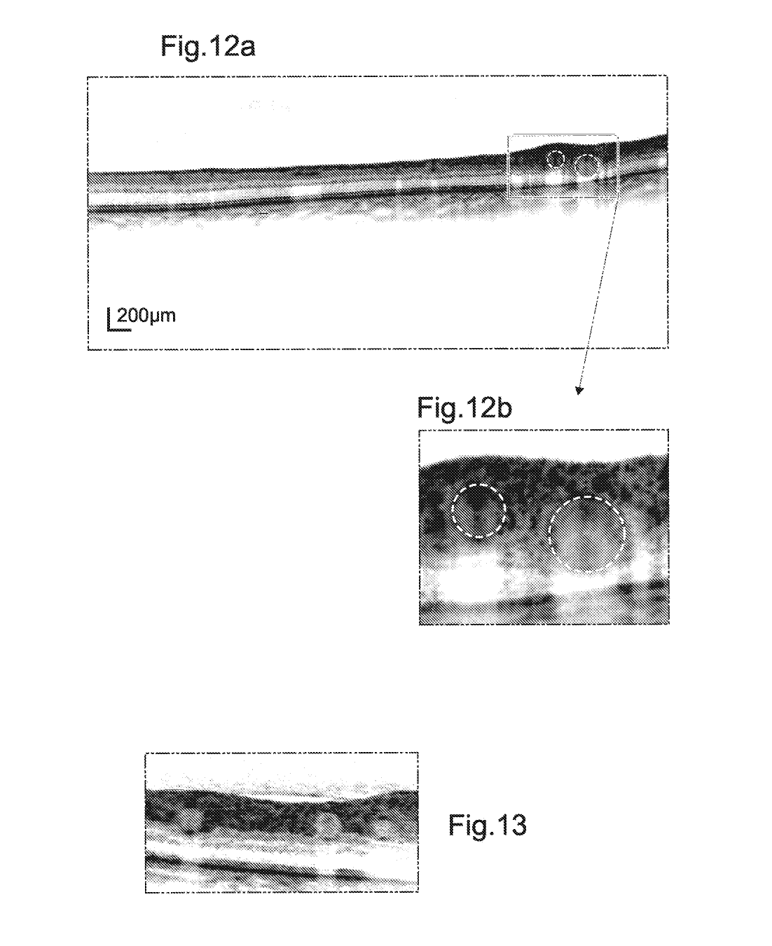

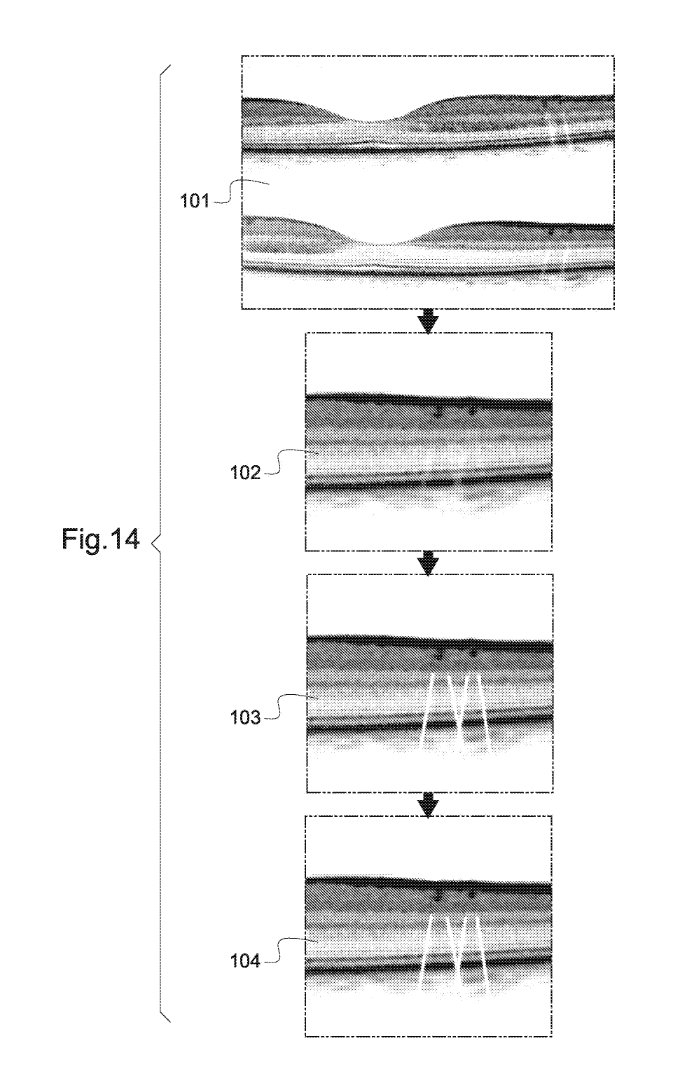

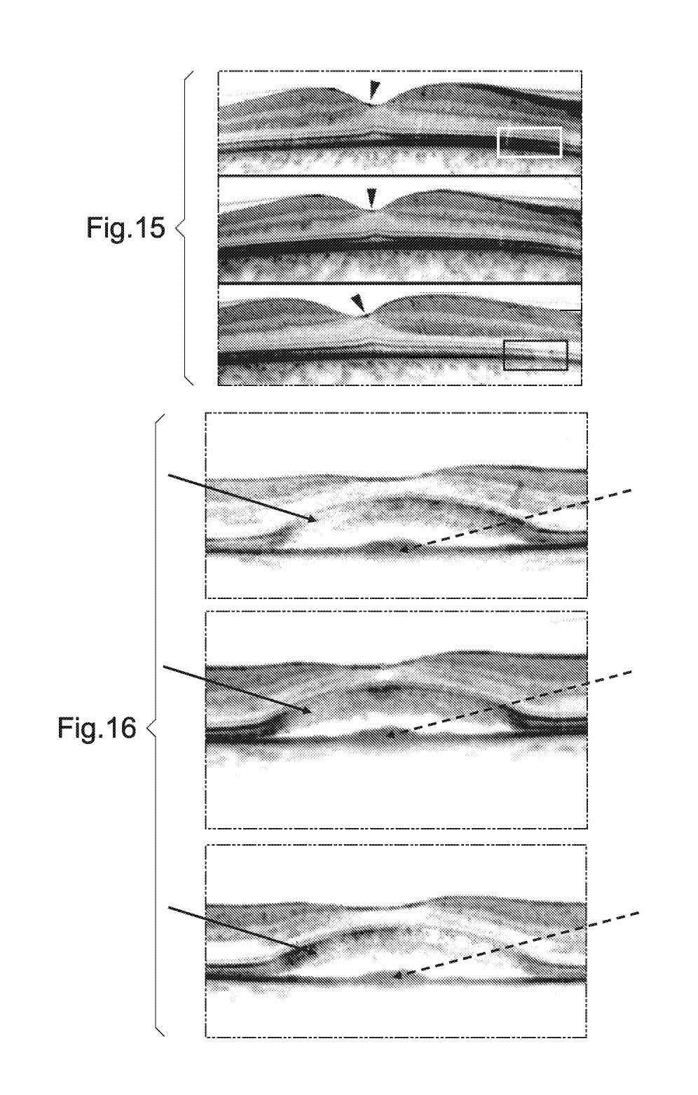

As was indicated above, certain structures of the retina take the form of fibrils that lie substantially parallel to one another, such as the axons of the photoreceptors, the axons of the ganglion cells, and the external segments of the photoreceptors, etc. Line-shaped patterns are liable to be due to the presence of these structures. The inventors have observed that a line-shaped pattern might even actually be one of these fibrils. With reference to FIG. 8, it may thus clearly be seen that the region framed by a white rectangle has a reflectance that differs depending on the angle of incidence with which a light beam (symbolized by the white arrow) passes through the retina. This region comprises external segments of the photoreceptors.

With reference to FIG. 9, a deposit (dotted arrow) is seen to have accumulated on the retina of a patient. Whereas, from one image to the other, the reflectance of the deposit remains the same, the reflectance of a pattern (solid arrow) due to external segments of the photoreceptors changes. It is thus possible and very easy to differentiate the deposit from the external segments.

Preferably, the device according to the invention comprises locating means. In a subsequent step, after the image in which the line is brightest has been selected, a direction in which said line extends is determined by way of locating means, in the image in which the line is brightest.

The inventors have been able to demonstrate that when the line is brightest, i.e. when its reflectance is highest, the direction of said line is liable to indicate a preferred direction of orientation of a structure taking the form of fibrils and located in a region adjacent said line. Determining the preferred direction of orientation thus allows the direction in which said fibrils are parallel on the whole to be determined.

The device and method according to the invention allow a large amount of information to be extracted from images taken by OCT. For a retinal tissue, it is thus especially possible to detect patterns that are liable to be due to the presence of blood vessels and fibrils, whether for a normal tissue or a diseased tissue.

Of course, the invention is not limited to the embodiment and implementation described and changes may be made thereto without departing from the scope of the invention as defined by the claims.

In particular, although here the device and method were described applied to a retinal tissue, the device and/or method of the invention may be employed to detect anatomical features in at least one portion of another tissue capable of being analyzed by OCT, such as a cornea, a mucosal tissue, or an epidermal portion for example. The device and/or the method will preferably be applied to tissues of small thickness, i.e. of a few millimeters in thickness at most. Hourglass-shaped patterns, or equally shadows, that are liable to be due to the presence of blood vessels may of course be detected in tissues other than a retinal tissue.

Although here only two images were acquired by OCT in order to aid with the detection of anatomical features in at least one portion of a tissue, more images will possibly be used. In addition, several patterns will possibly be detected at the same time, even if the patterns are all different.

1. A device for aiding with detection of anatomical features in at least one portion of a tissue, especially a retinal tissue, the device comprising means for acquiring, by optical coherence tomography (OCT), images of said portion taken at separate angles of incidence, and detecting means for detecting in the images, in a given region of the studied portion, at least one pattern having at least one characteristic that varies from one image to another, the pattern being liable to be due to the presence of anatomical features.

2. The device for aiding with detection as claimed in claim 1, in which the detecting means comprise means for searching for a pattern oriented differently from one image to another.

3. The device for aiding with detection as claimed in claim 2, the detecting means comprise means for searching for a pattern oriented differently from one image to another, which pattern is always oriented identically relative to a direction of a light beam that passes through the tissue for the purpose of image acquisition.

4. The device for aiding with detection as claimed in claim 3, in which the searching means comprise means for identifying an hourglass-shaped pattern.

5. The device for aiding with detection as claimed in claim 3, in which the searching means comprise means for identifying a pattern that is a shadow.

6. The device for aiding with detection as claimed in claim 5, furthermore comprising means for removing the shadow from a region that comprises the shadow by retrieving a section of said region, which section is not covered by the shadow, from each acquired image and superposing said sections.

7. The device for aiding with detection as claimed in claim 5, comprising means for locating a focal point by extending the shadows and identifying the point where they converge on the superposed images.

8. The device for aiding with detection as claimed in claim 1, in which the detecting means comprise means for searching for a pattern the reflectance of which is different from one image to another.

9. The device for aiding with detection as claimed in claim 8, comprising means for selecting the image in which the pattern is brightest.

10. The device for aiding with detection as claimed in claim 8, in which the searching means comprise means for identifying a line-shaped pattern.

11. The device for aiding with detection as claimed in claim 9, comprising means for locating a direction in which said line extends in the image in which the line is the brightest.

12. The device for aiding with detection as claimed in claim 1, furthermore comprising means for highlighting the pattern in one or more images.

13. The device for aiding with detection as claimed in claim 11, in which the highlighting means comprise means for encircling the pattern.

14. The device for aiding with detection as claimed in claim 12, in which the highlighting means comprise means for coloring the pattern.

15. A method for aiding with detection of anatomical features in at least one portion of a tissue, especially a retinal tissue, the method comprising steps of:
acquiring, by optical coherence tomography (OCT), at least two images of said portion taken at separate angles of incidence; and
detecting in the images, in a given region of the studied portion, at least one pattern having at least one characteristic that varies from one image to another, the pattern being liable to be due to the presence of anatomical features.
